(19) United States
(12) Patent Application Publication
(10) Pub. No.: US 2016/0135786 A1
(43) Pub. Date: May 19, 2016

(54) WIRELESS ULTRASOUND PROBE TETHERED TO A POD

(71) Applicant: General Electric Company, Schenectady, NY (US)

(72) Inventors: Paul Lawrence Mullen, Waukesha, WI (US); Roland Rott, Munich (DE)

(21) Appl. No.: 14/546,360

(22) Filed: Nov. 18, 2014

(51) Int. Cl. A61B 8/00; A61B 8/08

(52) U.S. Cl. CPC A61B 8/4472; A61B 8/4494; A61B 8/4433; A61B 8/546; A61B 8/4427; A61B 8/461; A61B 8/56; A61B 8/52

(57) ABSTRACT

An apparatus for wirelessly transmitting ultrasound image information includes an ultrasound probe including a transducer array, a transceiver to wirelessly communicate with an ultrasound console including a host processor and a display screen, a pod containing an electrical energy storage device to power the transducer array and the transceiver and a cable providing an electrical connection between the ultrasound probe and the pod.

### BACKGROUND

Ultrasound probes to aid in medical diagnoses and procedures typically are typically directly tethered to an ultrasound console by a cable. The console typically has a processor for giving direction to a transducer array in the ultrasound probe and for using the signal received from the transducer array to drive a display that is also a part of the console. The cable typical carries the directions to the transducer array in the ultrasound probe and carries the signal from this transducer array to the processor as well as supplying electrical energy to the ultrasound probe.

### SUMMARY

An embodiment involves an apparatus for wirelessly transmitting ultrasound image information. The apparatus includes an ultrasound probe including a transducer array, a transceiver to wirelessly communicate with an ultrasound console including a host processor and a display screen, a pod containing an electrical energy storage device to power the transducer array and the transceiver, and a cable providing an electrical connection between the ultrasound probe and the pod.

Another embodiment involves a medical diagnostic ultrasound system. It includes an ultrasound console with a processor that wirelessly receives ultrasound data and converts the ultrasound data it to a displayable image and a display screen on which the image may be displayed. The ultrasound system also includes an ultrasound probe with a transducer array, a transceiver to wirelessly communicate ultrasound data to the ultrasound console, a pod containing an electrical energy storage device to power the transducer array and the transceiver, and a cable providing an electrical connection between the ultrasound probe and the pod.

Another embodiment involves a process of taking a medical ultrasound image of a human patient by providing a medical diagnostic ultrasound system having an ultrasound console with a processor that wirelessly receives ultrasound data and converts the ultrasound data to a displayable image and a display screen on which the image may be displayed The system also includes an ultrasound probe comprising a transducer array, a transceiver to wirelessly communicate ultrasound data to the ultrasound console, a pod containing an electrical energy storage device to power the transducer array, and the transceiver and a cable providing an electrical connection between the ultrasound probe and the pod. The ultrasound probe and the pod are placed on one side of the patient and the ultrasound console on the other side of the patient such that the display of the console is in the line of sight of a sonographer taking the image while he is taking the image. Then the medical ultrasound image of the human patient is obtained using the ultrasound probe.

### BRIEF DESCRIPTION OF THE DRAWINGS

FIG. 1 is a schematic overview of a wireless ultrasound probe and cable connected pod.

FIG. 2 is a schematic representation of one embodiment of a medical ultrasound system with a wireless ultrasound probe and cable connected pod.

FIG. 3 is a flow chart of a process of wirelessly taking an ultrasound image.

FIG. 4 is a flow chart of a process of wirelessly transmitting commands and taking an ultrasound image.

### DETAILED DESCRIPTION

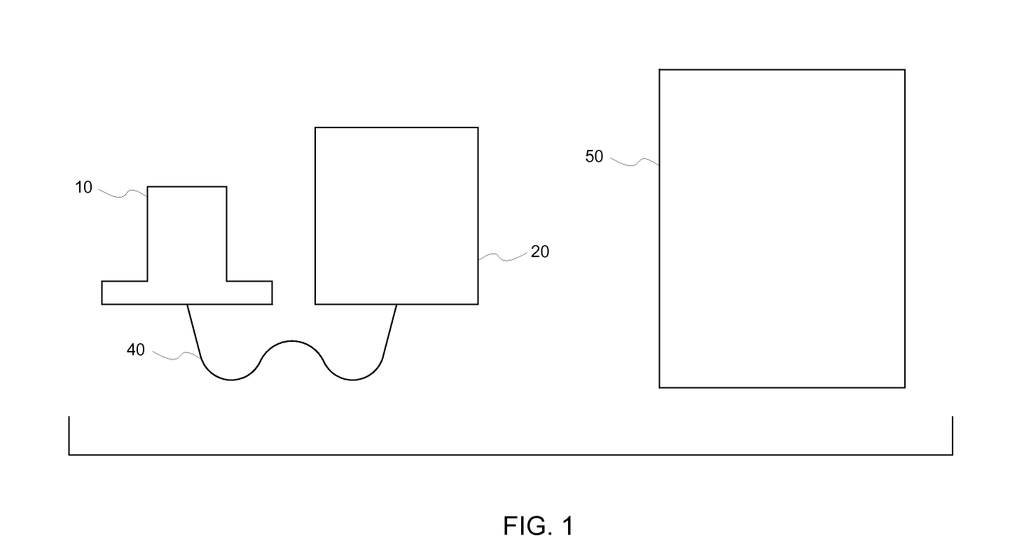

Referring to FIG. 1, an ultrasound probe 10 is connected to a pod 20 by a cable 40. This assemblage communicates wirelessly with an ultrasound console 50. The pod 20 enhances the capability of the ultrasound probe to function without a wired connection to the ultrasound console 50.

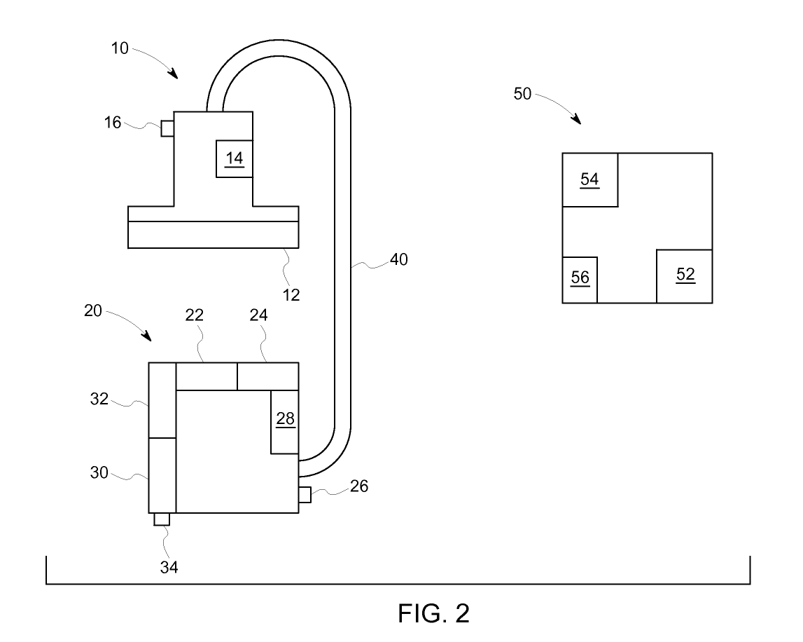

Referring to FIG. 2, an ultrasound probe 10 has a transducer array 12, a display monitor 14 and a control button 16. The display monitor 14 provides information to aid in the placement and movement of the ultrasound probe 10. In one embodiment display monitor 14 provides less information and a less detailed image than what is typically displayed on a full size ultrasound display monitor. In one embodiment display monitor 14 provides similar information and detail as a full size ultrasound display monitor. The control button 16 may be used to convey commands to an ultrasound console 50. The ultrasound probe 10 may also carry control elements by which the operation of the transducer array 12 may be modified locally. The ultrasound probe 10 may carry multiple buttons 16 or other control elements such as rotatable knobs or optical, capacitive or inertial sensors by which commands can be communicated to the ultrasound console 50. Because the ultrasound probe 10 has few key components probe 10 is lighter and more compact and generates less heat than if ultrasound probe 10 had more components.

A pod 20 has transceiver control circuitry 22 as well as a transceiver 24 with which to communicate with an ultrasound console 50. Pod 20 has a control button 26 which in one embodiment is used to convey commands to the ultrasound console 50. In one embodiment pod 20 includes multiple buttons 26 or other control elements such as rotatable knobs or optical, capacitive or inertial sensors by which commands can be communicated to the ultrasound console 50. The pod 20 may also carry control elements by which the operation of the transducer array 12 may be modified locally. The pod 20 also has a rechargeable battery 30 and battery management circuitry 32. The battery 30 also has a recharging connection 34, though in another embodiment the battery could be recharged by induction or the batteries could simply be readily replaceable. The pod 10 is of a size and weight such that it can be readily carried on the person of a sonographer using the ultrasound probe 10, such as in a pocket of the clothing of the sonographer. In one embodiment the pod 10 has the following dimensions: between 2 cm and 4 cm wide; 2 cm and 4 cm thick and 2 cm and 4 cm high. The pod 20 may conveniently carry most of the heat generating components typically involved in the operation of an ultrasound probe such as amplifiers, voltage converters, integrated circuits, and batteries.

A cable 40 provides an electrical connection between the ultrasound probe 10 and the pod 20. The cable 40 provides electrical energy from the battery 40 to the ultrasound probe transducer array 12 and to the ultrasound probe display monitor 14, as well as to any other function resident in the ultrasound probe 10 needing electrical energy. The cable 40 also provides for the transmission of signals between the pod transceiver 24 and the transceiver control circuitry 28 and the ultrasound probe transducer array 12, the ultrasound probe monitor display and the ultrasound probe control button 16. For instance, if the ultrasound probe control button 16 is activated a signal is provided via the cable 40 to the transceiver 24. The cable 40 also carries directions from the transducer array control circuitry 28 to the ultrasound probe transducer array 12 and carries any ultrasound signal received by this array 12 to this control circuitry 28 and the transceiver 24, as appropriate. In one embodiment cable 40 carries signals to drive the ultrasound probe display monitor 14. Cable 40 is of a flexibility, length and weight that it facilitates the sonographer having the pod 20 on his person, perhaps in a pocket of his clothing. In one embodiment cable 40 is readily detachable from the ultrasound probe 10, the pod 20 or both using a standardized connector such as a Universal Service Bus (USB) connector or a customized connector. In one embodiment data is communicated between the ultrasound probe 10 and the pod 20 via an optical cable. In one embodiment cable 40 is an electrical cable and in another embodiment, cable 40 transmits both optical and electrical signals. In one embodiment cable 40 has a length between about 25 cm and 200 cm. In one embodiment cable 40 has a length of between about 50 cm and 100 cm.

An ultrasound console 50 has a processor 52 which generates signals with which to control the ultrasound probe transducer array 12, receive signals back from this transducer array 12 and use these signals to drive a display monitor 54 which is a part of the ultrasound console 50. The processor 52 is also responsive to the control signal from the ultrasound probe control button 16 and the pod control button 26. An ultrasound console transceiver 56 serves to wirelessly send signals and receive signals from the pod transceiver 24. The console may conveniently be an off-the-shelf or consumer electronic device such as a tablet, smartphone or personal computer (PC) of the desktop or lap top variety with an appropriate application (app) or it may be a dedicated device. In some embodiments all the image processing is handled by components in the ultrasound probe 10 and the pod 20 and completed images are streamed to the console 50, which then displays the images. In other embodiments the console 50 may have the facility to transmit commands to the ultrasound probe 10 and the pod 20. In one embodiment console 50 has a touch screen type user interface.

For purposes of this application, the term “processing unit” or “processor” shall mean a presently developed or future developed processing unit that executes sequences of instructions contained in a memory. Execution of the sequences of instructions causes the one or more processing units to perform steps such as generating control signals. The instructions may be loaded in a random access memory (RAM) for execution by the one or more processing units from a read only memory (ROM), a mass storage device, or some other persistent storage. In other embodiments, hardwired circuitry may be used in place of or in combination with software instructions to implement the functions described. For example, the functionality of ultrasound probe 10 may be implemented entirely or in part by one or more application-specific integrated circuits (ASICs). Unless otherwise specifically noted, the ultrasound console is not limited to any specific combination of hardware circuitry and software, nor to any particular source for the instructions executed by the one or more processing units.

For purposes of this application memory includes a non-transient computer-readable medium or other persistent storage device, volatile memory such as DRAM, or some combination of these; for example a hard disk combined with RAM. Memory contains instructions for directing the carrying out of functions and analysis by one or more processors. In some implementations, memory further stores data for use by the one or more processors. Memory stores various software or code modules that direct the processor to carry out various interrelated actions.

The combination of the ultrasound probe 10, the pod 20 and the cable 40 allows for the arbitrary arrangements of the ultrasound system so that the user may readily place the console, patient, probe, and himself in a way that is ergonomically healthy, or in a way that facilitates a comfortable or efficient completion of the ultrasound procedure. This may include things like the operator placing the ultrasound console display in his line of sight while using the ultrasound probe 10 to acquire an image from a human patient. One embodiment involves having the sonographer on one side of the patient and the ultrasound console 50 on the other side of the patient. Because the ultrasound probe 10 is not tethered to the ultrasound console 50 the physical relationship between them is less constrained.

On the other hand, because space and weight consuming and heat generating functions have been moved from the ultrasound probe 10 to the pod 20 the quality of the ultrasound data and the capability of the transducer array is also less constrained. Thus more image-formation and control circuitry can be placed in the ultrasound probe 10 without it becoming unacceptably large or heavy.

It is convenient to configure the probe 10, the pod 20 and the cable 40 to provide a portion that may be worn on a body part of the user. For instance the cable 40 may be configured as a lanyard to fit about the neck or wrist of the user. In one embodiment the pod 20 includes an attachment device such as a neck lanyard or wrist strap.

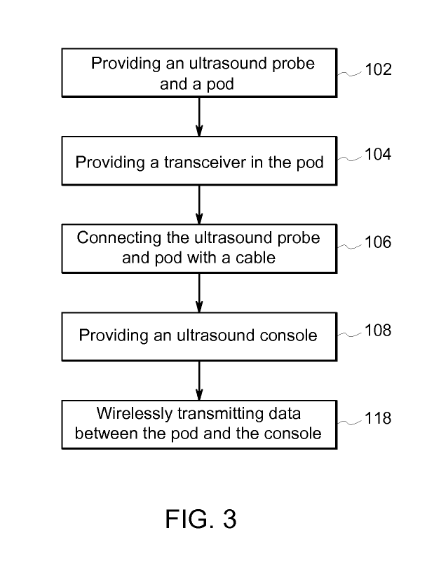

Referring to FIG. 3, a process of acquiring an ultrasound image involves a step 102 of providing an ultrasound probe and a pod, a step 104 of providing a transceiver in the pod, a step 106 of connecting the ultrasound probe and the pod with a cable, a step 108 of providing an ultrasound console and a step 118 of wirelessly transmitting data between the pod and the console. The wireless data transmission may be in accordance with any standard data transmission protocol and technique including but not limited to WiFi and Bluetooth.

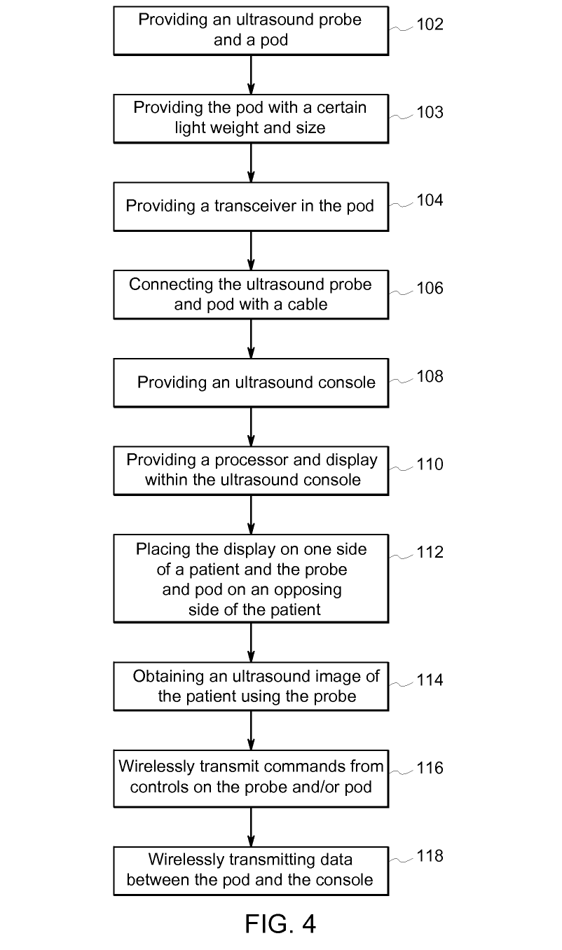

Referring to FIG. 4, a process of acquiring an ultrasound image involves the same steps 102-108 as in the process of FIG. 3 but also involves a step 110 of providing a processor and display within the ultrasound console, a step 112 of placing the display on one side of a patient and the probe and pod on an opposing side of the patient, a step 114 of obtaining an ultrasound image of the patient and a step 116 to wirelessly transmit commands from controls on the probe and/or pod and then the same step 118 of wirelessly transmitting data between the pod and the console. The wireless command transmission may be in accordance with any standard data transmission protocol and technique including WiFi and Bluetooth. In fact, current Bluetooth may be more appropriate for command transmission than data transmission given its current data transmission capabilities.

Although the present disclosure has been described with reference to example embodiments, workers skilled in the art will recognize that changes may be made in form and detail without departing from the spirit and scope of the claimed subject matter. For example, although different example embodiments may have been described as including one or more features providing one or more benefits, it is contemplated that the described features may be interchanged with one another or alternatively be combined with one another in the described example embodiments or in other alternative embodiments. Because the technology of the present disclosure is relatively complex, not all changes in the technology are foreseeable. The present disclosure described with reference to the example embodiments and set forth in the following claims is manifestly intended to be as broad as possible. For example, unless specifically otherwise noted, the claims reciting a single particular element also encompass a plurality of such particular elements.

What is claimed is:

1. An apparatus for wirelessly transmitting ultrasound image information comprising:
an ultrasound probe including a transducer array;
a transceiver to wirelessly communicate with an ultrasound console including a host processor and a display screen;
a pod containing an electrical energy storage device to power the transducer array and the transceiver; and
a cable providing an electrical connection between the ultrasound probe and the pod.

2. The apparatus of claim 1 wherein the pod contains the circuitry to control the transceiver.

3. The apparatus of claim 2 wherein the transceiver is located in the pod.

4. The apparatus of claim 1 wherein the pod has dimensions and a weight conducive to being carried in the pocket of a sonographer using the ultrasound probe on a patient.

5. The apparatus of claim 3 wherein the cable has a flexibility and length conducive to the pod being carried in the pocket of a sonographer using the ultrasound probe on a patient.

6. The apparatus of claim 1 wherein pod contains the control circuitry to control the transducer array.

7. The apparatus of claim 1 wherein the ultrasound probe carries a small display monitor.

8. The apparatus of claim 1 wherein the pod contains the circuitry to manage the electrical energy storage device.

9. The apparatus of claim 1 wherein the electrical energy storage device is a battery.

10. The apparatus of claim 9 wherein the battery is rechargeable.

11. The apparatus of claim 1 wherein the pod is separate from and not secured to the ultrasound probe by other than the cable.

12. The apparatus of claim 1 wherein the ultrasound probe carries controls that cause commands to be wirelessly transmitted to an ultrasound console comprising a host computer and display screen.

13. The apparatus of claim 1 wherein the pod carries controls that cause commands to be wirelessly transmitted to an ultrasound console comprising a host computer and display screen.

14. The apparatus of claim 1 wherein the ultrasound probe or the pod carries controls that alter the manner in which data from the transducer array is processed.

15. The apparatus of claim 1 wherein the pod carries controls that alter the manner in which transducer array is operated.

16. The apparatus of claim 1 wherein the pod contains all the heat generating components necessary to operate the transducer array and wirelessly transmit the ultrasound data obtained from the transducer array, other than the transducer array itself.

17. A medical diagnostic ultrasound system comprising:
an ultrasound console comprising a processor that wirelessly receives ultrasound data and converts it to a displayable image and a display screen on which the image may be displayed;
an ultrasound probe comprising a transducer array;
a transceiver to wirelessly communicate ultrasound data to the ultrasound console;
a pod containing an electrical energy storage device to power the transducer array and the transceiver; and
a cable providing a connection between the ultrasound probe and the pod.

18. The medical diagnostic ultrasound system of claim 17 wherein:
the transceiver is located in the pod;
the pod has dimensions and a weight and the cable has a flexibility and length conducive to the pod being carried in the pocket of a sonographer using the ultrasound probe on a patient; and
either or both of the ultrasound probe or the pod carry controls that cause commands to be wirelessly transmitted to the ultrasound console.

19. A process of taking a medical ultrasound image of a human patient comprising:
providing a medical diagnostic ultrasound system having;
an ultrasound console comprising a processor that wirelessly receives ultrasound data and converts it to a displayable image and a display screen on which the image may be displayed;
an ultrasound probe comprising a transducer array;
a transceiver to wirelessly communicate ultrasound data to the ultrasound console;
a pod containing an electrical energy storage device to power the transducer array and the transceiver; and
a cable providing an electrical connection between the ultrasound probe and the pod;
placing the ultrasound probe and pod on one side of the patient and the ultrasound console on the other side of the patient such that the display of the console is in the line of sight of a sonographer taking the image while he is taking the image;
obtaining the medical ultrasound image of the human patient using the ultrasound probe.

20. The process of claim 19 wherein:
the pod has dimensions and a weight and the cable has a flexibility and length conducive to the pod being carried in the pocket of a sonographer using the ultrasound probe on a patient; and
one or both of the ultrasound probe and the pod carry controls that cause commands to be wirelessly transmitted to the ultrasound console.
